(19) United States
(12) Patent Application Publication
(10) Pub. No.: US 2004/0043033 A1
(43) Pub. Date: Mar. 4, 2004

(54) Method and vaccine for the prevention of AIDS

(76) Inventor: Lorrence H. Green, Westbury, NY (US)

Correspondence Address: Thomas A. O'Rourke, Bodner & O'Rourke, 425 Broadhollow RD, Melville, NY, 11747, US

(21) Appl. No.: 09/846,687

(22) Filed: May 1, 2001

Related U.S. Application Data
(60) Provisional application No. 60/200,983, filed on May 1, 2000.

(51) Int. Cl. A61K 39/00

(52) U.S. Cl. 424/185.1

(57) ABSTRACT

The present invention is directed to a method of inducing the body to produce an antibody against the region of the CCR5 receptor in wild type individuals, that is affected by the delta 32 deletion and vaccines for producing said antibody. The antibody is produced is by treating the individual using a vaccine consisting of a polypeptide and its derivatives.

The present invention is based on U.S. Provisional Application No. 60/200,983, filed on May 1, 2000, the disclosures of which are incorporated herein by reference.

### FIELD OF THE INVENTION

The present invention relates to a method of treating individuals infected with HIV to reduce the risk the HIV infection will lead to AIDS.

### BACKGROUND OF THE INVENTION

In the last twenty or so years there has been a great deal of effort in attempting to understand and find a cure for AIDS. The presence of the human immunodeficiency virus (HIV) in many instances induces a persistent and progressive infection. In the vast majority of cases the infection leads to the development of the acquired immunodeficiency syndrome (AIDS). In humans, HIV replication occurs prominently in CD4.sup.+ T lymphocyte populations, and HIV infection leads to depletion of this cell type and eventually to immune incompetence, opportunistic infections, neurological dysfunctions, neoplastic growth, and ultimately death.

HIV infection is pandemic and HIV-associated diseases represent a major world health problem. Although considerable effort is being put into the design of effective therapeutics, currently no curative anti-retroviral drugs against AIDS exist. In attempts to develop such drugs, several stages of the HIV life cycle have been considered aLs targets for therapeutic intervention. Many viral targets for intervention with the HIV life cycle have been suggested, as the prevailing view is that interference with a host cell protein would have deleterious side effects. For example, virally encoded reverse transcriptase has been one focus of drug development. A number of reverse-transcriptase-targeted drugs, including 2′,3′dideoxynucleoside analogs such as AZT, ddI, ddc, and d4T have been developed which have been shown to been active against HIV.

The new treatment regimens for HIV-1 show that a combination of anti-HIV compounds, which target reverse transcriptase (RT), such as azidothymidine (AZT), lamivudine (3TC), dideoxyinosine (ddi), dideoxycytidine (ddc) used in combination with an HIV-1 protease inhibitor have a far greater effect (2 to 3 logs reduction) on viral load compared to AZT alone (about 1 log reduction). For example, impressive results have recently been obtained with a combination of AZT, ddI, 3TC and ritonavir. However, it is likely that long-term use of combinations of these chemicals will lead to toxicity, especially to the bone marrow. Long-term cytotoxic therapy may also lead to suppression of CD8.sup.+ T cells, which are essential to the control of HIV, via killer cell activity and by the release of factors which inhibit HIV infection or replication, notably the chemokines Rantes, MIP-1.alpha. and MIP-1.beta. (Cocchi et al., 1995, Science 270:1811-1815). Another major concern in long-term chemical anti-retroviral therapy is the development of HIV mutations with partial or complete resistance. It is thought that such mutations may be an inevitable consequence of anti-viral therapy. The pattern of disappearance of wild-type virus and appearance of mutant virus due to treatment, combined with coincidental decline in CD4.sup.+ T cell numbers strongly suggests that, at least with some compounds, the appearance of viral mutants is a major underlying factor in the failure of AIDS therapy.

HIV is a member of the lentivirus family of retroviruses. Retroviruses are small enveloped viruses that contain a single-stranded RNA genome, and replicate via a DNA intermediate produced by a virally-encoded reverse transcriptase, an RNA-dependent DNA polymerase. The HIV viral particle consists of a viral core, composed in part of capsid proteins designated p24 and p18, together with the viral RNA genome and those enzymes required for early replicative events. Myristylated gag protein forms an outer viral shell around the viral core, which is, in turn, surrounded by a lipid membrane envelope derived from the infected cell membrane. The HIV envelope surface glycoproteins are synthesized as a single 160 kilodalton precursor protein which is cleaved by a cellular protease during viral budding into two glycoproteins, gp41 and gp120. gp41 is a transmembrane glycoprotein and gp120 is an extracellular glycoprotein which remains non-covalently associated with gp41, possibly in a trimeric or multimeric form.

HIV, like other enveloped viruses, introduces viral genetic material into the host cell through a viral-envelope mediated fusion of viral and target membranes. HIV is targeted to CD4.sup.+ cells because a CD4 cell surface protein (CD4) acts as the cellular receptor for the HIV-1 virus. Viral entry into cells is dependent upon gp120 binding the cellular CD4 receptor molecules, explaining HIV's tropism for CD4.sup.+ cells, while gp41 anchors the envelope glycoprotein complex in the viral membrane. The binding of gp120 to CD4 induces conformational changes in the viral glycoproteins, but this binding alone is insufficient to lead to infection.

Studies of HIV-1 isolates have revealed a heterogeneity in their ability to infect different human cell types. The majority of extensively passaged laboratory strains of HIV-1 readily infect cultured T cell lines and primary T lymphocytes, but not primary monocytes or macrophages. These strains are termed T-tropic. T-tropic HIV-1 strains are more likely to be found in HIV-1 infected individuals during the late stages of aids. The majority of primary HIV-1 isolates (i.e., viruses not extensively passaged in culture) replicate efficiently in primary lymphocytes, monocytes and macrophages, but grow poorly in established T cell lines. These isolates have been termed M-tropic. The viral determinant of T- and M-tropism maps to alterations in the third variable region of gp120 (the V3 loop). The characterization of HIV isolates with distinct tropisms taken together with the observation that binding to the CD4 cell surface protein alone is insufficient to lead to infection, suggest that cell-type specific cofactors might be required in addition to CD4 for HIV-1 entry into the host cell.

The late stages of HIV replication, which involve crucial virus-specific processing of certain viral encoded proteins, have been suggested as possible anti-HIV drug targets. Late stage processing is dependent on the activity of a viral protease, and drugs are being developed which inhibit this protease. Attention is also being given to the development of vaccines for the treatment of HIV infection. The HIV-1 envelope proteins (gp160, gp1120, gp41) have been shown to be the major antigens for anti-HIV antibodies present in AIDS patients.

Although human immunodeficiency virus type-1 (“HIV-1”) uses the T cell surface molecule CD4 as a primary receptor, successful viral entry into and infection of a cell has been found to require the presence of a second molecule, or “co-receptor” (Clapham and Weiss, 1997, Nature 388:230-231). Seven co-receptor molecules have been identified, each of which are members of, or related to, the family of chemokine receptors, which are G-protein coupled receptors having seven transmembrane domains.

Recently, certain chemokines produced by CD8.sup.+ T cells have been implicated in suppression of HIV infection. The chemokines RANTES (regulated on activation normal T cell expressed and secreted), macrophage-inflammatory protein-1.alpha. and -1.beta. (MIP-1.alpha. and MIP-1.beta., respectively), which are secreted by CD8.sup.+ T cells, have been shown to suppress HIV-1 p24 antigen production in cells infected with HIV-1 or HIV-2 isolates in vitro. Additionally, high levels of these chemokines have been found to be secreted by CD4.sup.+ T lymphocytes in individuals that have been exposed to HIV-1 on multiple occasions but, remain uninfected (Paxton et al., 1996, Nature Med. 2:412-417). While RANTES, MIP-1.alpha. and MIP-1.beta. alone or in combination, potently suppress a variety of primary HIV-1 isolates and macrophage tropic isolates, such as HIV-1.sub.BaL, some established laboratory strains, such as HIV-1. sub.IIIB, are refractory to inhibition of infection or replication by these chemokines (Cocchi et al., 1995, Science 270:1811-1815).

Chemokines, or chemoattractant cytokines, are a subgroup of immune factors that have been shown to mediate chemotactic and other pro-inflammatory phenomena. Chemokines are small molecules of approximately 70-80 residues in length and can generally be divided into two subgroups, alpha. which have two N-terminal cysteines separated by a single amino acid (CxC) and beta. which have two adjacent cysteines at the N terminus (CC). RANTES, MIP-1.alpha. and MIP-1.beta. are members of the .beta. subgroup. The amino terminus of the .beta. chemokines RANTES, MCP-1, and MCP-3 have been implicated in the mediation of cell migration and inflammation induced by these chemokines. This involvement is suggested by the observation that the deletion of the amino terminal 8 residues of MCP-1, amino terminal 9 residues of MCP-3, and amino terminal 8 residues of RANTES and the addition of a methionine to the amino terminus of RANTES, antagonize the chemotaxis, calcium mobilization and/or enzyme release stimulated by their native counterparts. Additionally, alpha. chemokine-like chemotactic activity has been introduced into MCP-1 via a double mutation of Tyr 28 and Arg 30 to leucine and valine, respectively, indicating that internal regions of this protein also play a role in regulating chemotactic activity.

The monomeric forms of all chemokines characterized typically share significant structural homology, although the quaternary structures of alpha. and .beta. groups are distinct. While the monomeric structures of the beta. and alpha. chemokines are very similar, the dimeric structures of the two groups are completely different. An additional chemokine, lymphotactin, which has only one N terminal cysteine has also been identified and may represent an additional subgroup (.gamma.) of chemokines.

Receptors for chemokines belong to the large family of G-protein coupled, 7 transmembrane domain receptors (GCR's). Competition binding and cross-desensitization studies have shown that chemokine receptors exhibit considerable promiscuity in ligand binding. Examples demonstrating the promiscuity among beta. chemokine receptors include: CC CKR-1, which binds RANTES and MIP-1.alpha., CC CKR-4, which binds RANTES, MIP-1.alpha., and MCP-1, and CC CKR-5, which binds RANTES, MIP-1.alpha., and MIP-1.beta. Erythrocytes possess a receptor (known as the Duffy antigen) which binds both alpha. and beta. chemokines. Thus the sequence and structural homologies evident among chemokines and their receptors allows some overlap in receptor-ligand interactions.

CC CKR-5 is the major coreceptor for macrophage-tropic strains of HIV-1. RANTES, MIP-1.alpha., or MIP-1.beta., the chemokine ligands for this receptor have been shown to block HIV Env-mediated cell fusion directed by CC CKR-5. Additional support for the role of CC CKR-5 as an M-tropic HIV-1 cofactor comes from the finding that a 32-base pair deletion in the CC CKR-5 gene found in three multiply exposed but uninfected individuals, prevents HIV from infecting macrophages. However, only three of the 25 uninfected individuals studied had this mutation.

The V3 loop of gp120 is the major determinant of sensitivity to chemokine inhibition of infection or replication. Signal transduction through .beta. chemokine receptors is not required for inhibition of HIV infection or replication, since RANTES inhibits HIV-1 infection in the presence of pertussis toxin, an inhibitor of G-protein-mediated signaling pathways. CxC CKR4, a CxC (.alpha.) chemokine receptor, has been shown to be a coreceptor involved in infection by laboratory-adapted HIV-1 strains. The alpha. chemokine SDF-1, the ligand for this receptor, has been demonstrated to block infection by T-tropic HIV-1 isolates. CxC CKR4 does not bind the beta chemokines RANTES, MIP-1.alpha., or MIP-1.beta.

Recently, it has been shown that certain primary, syncytium-inducing/T-tropic isolates use both CC CKR5 and CxC CKR4 as coreceptors and are able to switch between the two. Thus, in the presence of RANTES, MIP-1 alpha. and MIP-1.beta., the chemokine ligands for CC CKR5, T-tropic strains are still able to infect cells via the CxC CKR4 coreceptor. Attempts are also being made to develop drugs which can inhibit viral entry into the cell, the earliest stage of HIV infection, by focusing on CD4, the cell surface receptor for HIV. Recombinant soluble CD4, for example, has been shown to inhibit infection of CD4.sup.+ T cells by some HIV-1 strains. Certain primary HIV-1 isolates, however, are relatively less sensitive to inhibition by recombinant CD4. In addition, recombinant soluble CD4 clinical trials have produced inconclusive results.

Two species of chemokine receptors which appear to be particularly relevant to HIV infection are CCR5 and CXCR4, for which the natural ligands are MIP-1a, MIP-1b and RANTES (CCR5) and SDF-1 (CXCR4). To date, most HIV-1 clinical isolates appear to use CCR5 or CXCR4, or both, as co-receptors with CD4 for entry into cells (“Dynamics of HIV Infection”, Science and Medicine, March/April 1998: 36-45), and the presence of chemokine ligand inhibits infection via the corresponding receptor.

The cellular distributions of CCR5 and CXCR4 are associated with the role of these molecules in the course of HIV-1 infection. CCR5, which is mainly expressed on macrophages and memory T cells, serves as a co-receptor for infection by macrophage-tropic (“M-tropic”) strains of HIV-1, which are found throughout the course of infection, are preferentially involved in sexual transmission of HIV-1, and are represented by non-syncytium-inducing laboratory isolates which do not cause cell/cell fusion in T cell lines. CXCR4, however, which is expressed on a broader spectrum of cells, including naive T cells, serves as the co-receptor in late stages of infection for syncytium-inducing, T-cell-tropic (“T-tropic”) strains of HIV-1. Accordingly, the co-receptor which is more relevant to the initiation of HIV-1 infection appears to be CCR5.

An association has been drawn between those rare individuals who remain persistently uninfected despite multiple sexual exposures to HIV and the presence of a 32 base pair deletion in the CCR5 gene (“CCR5.DELTA.32”; Samson et al., 1996, Nature 382:722-725; Liu et al., 1996, Cell 86:367) which results in a frame shift mutation and the loss of the last three of the seven transmembrane domains (including the fifth, sixth and seventh transmembrane domains) present in the wild-type protein. Individuals heterozygous for this deletion, are, however, susceptible to infection (Dean et al., Science 273:1856), although progression to AIDS may be slowed (Dean et al., 1996, Science 273:1856-1862; Samson et al., 1996, Nature 382:722-725; Huang et al., 1996, Nature Med. 2:1240-1243; Michael et al., 1997, Nature Med. 3:338-340). It has been proposed (Benkirane et al., December 1997, J. Biol. Chem. 272:30603-30606) that co-expression of the CCR5.DELTA.32 gene with the wild-type CCR5 gene results in trans-inhibition of the ability of CCR5 to act as an HIV co-receptor, in which the CCR5.DELTA.32 protein interferes with dimerization of CCR5 at the cell surface. It has not, however, been confirmed that dimerization of CCR5 occurs or is necessary for viral entry.

### SUMMARY OF THE INVENTION

The present invention is directed to a method of inducing the body to produce an antibody against the region of the CCR5 receptor in wild type individuals, that is affected by the delta 32 deletion and vaccines for producing said antibody. The antibody is produced is by treating the individual using a vaccine consisting of the following polypeptide and derivatives thereof:

Y-S-Q-Y-Q-F-W-K-N-F-Q-T-LK-I-V-I-L-G-L-V-L-P-L-L-
V-M-V-I-C-Y-S-G-I-L-K-T-L-L-R-C-R-N-EK-K-R (Tyr-
Ser-Gln-Tyr-Gln-Phe-Trp-Lys-Asn-Phe-Gln-Thr-Leu-
LysIle-Val-Ile-Leu-Gly-Leu-V al-Leu-Pro-Leu-Leu-
V al-Met-Val-Ile-Cys-TyrSer-Gly-Ile-Leu-Lys-Thr-
Leu-Leu-Arg-Cys-Arg-Asn-Glu-Lys-Lys-Arg).

The polypeptide based vaccine of the present invention contains the amino acids that are present in the wild type CCR5 receptor, that are eliminated or replaced in the delta 32 deletion. Using this molecule (or a derivative of it), the body will produce an antibody, that binds to the CCR5 receptor.

The present invention also has applicability in the treatment of other conditions since polypeptides can be used as a mechanism of causing the body to generate other antibodies, that can specifically inactivate viral receptors. More particularly, the teachings of the present invention will also apply to any disease in which specifically inactivating the viral binding site is not deleterious to the body. Inactivating the active viral binding site without causing deleterious effects has clearly been demonstrated m the case of CCR5 and HIV The uniqueness of this (these) compound(s), and this methodology is that the body's own defense mechanisms, produce antibodies to “inactivate” a part of the body that has been exploited by a pathogen. The compound(s) are unlike other anti-infective treatments, in that they are not directly destroying the pathogen. They are inducing the body to destroy the pathogens ability to infect.

### DETAILED DESCRIPTION OF THE INVENTION

The present invention is directed to a method of inducing the body of an individual, typically an individual infected with HIV to produce an antibody against the region of the CCR5 receptor in wild type individuals, that is affected by the delta 32 deletion. People homozygous for the delta 32 deletion of the CCR5 chemokine receptor have been found to be immune to infection by Human Immunodeficiency Virus (HIV). People with these deletions are known to have CCR5 chemokine receptor molecules in which some amino acids are missing, and others have been replaced. Having mutant CCR5 receptors, does not appear to have a deleterious effect upon the health of those individuals that have the deletion.

The treatment of the present invention entails the inducing of the body to produce an antibody against the region of the CCR5 receptor, in wild type individuals, that is effected by the delta 32 deletion. This is accomplished by using a vaccine consisting of the following polypeptide:

Y-S-Q-Y-Q-F-W-K-N-F-Q-T-LK-I-V-I-L-G-L-V-L-P-L-L-
V-M-V-I-C-Y-S-G-I-L-K-T-L-L-R-C-R-N-E-K-K-R (Tyr-
Ser-Gln-Tyr-Gln-Phe-Trp-Lys-Asn-Phe-Gln-Thr-Leu-
LysIle-Val-Ile-Leu-Gly-Leu-Val-Leu-Pro-Leu-Leu-
Val-Met-Val-Ile-Cys-TyrSer-Gly-Ile-Leu-Lys-Thr-
Leu-Leu-Arg-Cys-Arg-Asn-Glu-Lys-Lys-Arg).

Derivatives of the polypeptide may include compounds in which any one or more of the amino acids in the invention has been substituted with one of similar charge, acidity, basicity, structure or functional group. For instance in the initial amino acid sequence Tyr-Ser-Tyr-Gln, Serine (Ser) is an amino acid containing a hydroxyl group. Threonine (Thr) is also a hydroxyl group containing amino acid. A derivative of the polypeptide would include Tyr-Thr-Tyr-Gln, in which one hydroxyl containing amino acid is substituted for another. Likewise in the sequence Leu-Leu-Val-Met-Val, Methionine (Met) is a sulfur containing side chain. A derivative of this would include Leu-Leu-Val-Cys-Val, where the sulfur containing amino acid Cysteine (Cys) is substituted for the sulfur containing Met. Likewise derivatives may also include chemical modifications of the amino acids described in this invention. For instance, many of the amino acids can be methylated, hydrated, or modified using several other standard chemical methods. These modified amino acids would also be considered derivatives of the invention. This invention also includes the three dimensional structure that is generated by this amino acid sequence. It is possible that a molecule with the same three dimensional structure of the invention, can be generated using a completely different set of amino acids. This new molecule would also be considered a derivative of this invention.

The polypeptide based vaccine of the present invention contains the amino acids that are present in the wild type CCR5 receptor, that are eliminated or replaced in the delta 32 deletion. Using this molecule (or a derivative of it), the body will produce an antibody, that binds to the CCR5 receptor. With an antibody bound to the CCR5 site, the ability of HIV to gain entrance to the cell will be prevented, or at least inhibited long enough, for the body's natural defenses to destroy it. The vaccine of the present invention may use of part or all of this molecule as a hapten, and use the different three dimensional configurations of this molecule.

It has been shown that throughout the long “incubation phase” of an HIV infection, the body is actually destroying virally infected CD4 cells, and producing new ones. This compound, if administered early in the infection, will also prevent early active infections from overwhelming the body, and will be an effective treatment. The antibodies produced by the compound will bind to the CCR5 receptor on the new cells that the body is producing, and prevent HIV from entering them.

This treatment represents only one example of the concept of using polypeptides as a mechanism of causing the body to generate antibodies, that can specifically inactivate viral receptors. It will apply to any disease in which specifically inactivating the viral binding site. is not deleterious to the body.

The uniqueness of this (these) compound(s), and this methodology is that the body's own defense mechanisms, produce antibodies to “inactivate” a part of the body that has been exploited by a pathogen. The compound(s) are unlike other anti-infective treatments, in that they are not directly destroying the pathogen. They are inducing the body to destroy the pathogens ability to infect.

1. A method of inducing the body to produce an antibody against the region of the CCR5 receptor in wild type individuals, that is affected by the delta 32 deletion comprising using a vaccine including a polypeptide having the following sequence:
Tyr-Ser-Gln-Tyr-Gln-Phe-Trp-Lys-Asn-Phe-Gln-Thr-
Leu-LysIle-V al-Ile-Leu-Gly-Leu-V al-Leu-Pro-Leu-
Leu-V al-Met-V al-Ile-Cys-TyrSer-Gly-Ile-Leu-Lys-
Thr-Leu-Leu-Arg-Cys-Arg-Asn-Glu-Lys-Lys-Arg.

2. The method according to claim 1 wherein the vaccine is a derivative of said polypeptide.

3. The method according to claim 1 wherein said vaccine produces an antibody bound to the CCR5 site.

4. A method of treating a patient infected with HIV comprising using a vaccine including a polypeptide having the following sequence:
Tyr-Ser-Gln-Tyr-Gln-Phe-Trp-Lys-Asn-Phe-Gln-Thr-
Leu-LysIle-V al-Ile-Leu-Gly-Leu-Val-Leu-Pro-Leu-
Leu-Val-Met-V al-Ile-Cys-TyrSer-Gly-lle-Leu-Lys-
Thr-Leu-Leu-Arg-Cys-Arg-Asn-Glu-Lys-Lys-Arg
wherein said vaccine produces an antibody against the region of the CCR5 receptor in wild type individuals, that is affected by the delta 32 deletion.

5. The method according to claim 2 wherein the vaccine is a derivative of said polypeptide.

6. A vaccine for producing an antibody against the region of the CCR5 receptor in wild type individuals, that is affected by the delta 32 deletion comprising a polypeptide having the following sequence:
Tyr-Ser-Gln-Tyr-Gln-Phe-Trp-Lys-Asn-Phe-Gln-Thr-
Leu-LysIle-Val-Ile-Leu-Gly-Leu-V al-Leu-Pro-Leu-
Leu-Val-Met-Val-Ile-Cys-TyrSer-Gly-Ile-Leu-Lys-
Thr-Leu-Leu-Arg-Cys-Arg-Asn-Glu-Lys-Lys-Arg.

7. A method of vaccination comprising providing a polypeptide that causes a body to generate antibodies in response to said polypeptide, said antibodies inactivating viral receptors.

8. The method according to claim 7 wherein said polypeptide has the following sequence:
Tyr-Ser-Gln-Tyr-Gln-Phe-Trp-Lys-Asn-Phe-Gln-Thr-
Leu-LysIle-Val-Ile-Leu-Gly-Leu-V al-Leu-Pro-Leu-
Leu-Val-Met-Val-IIe-Cys-TyrSer-Gly-Ile-Leu-Lys-
Thr-Leu-Leu-Arg-Cys-Arg-Asn-Glu-Lys-Lys-Arg.

9. The method according to claim 8 wherein the vaccine is a derivative of said polypeptide.

10. The method according to claim 9 wherein said vaccine produces an antibody bound to the CCR5 site.
